(19) United States
(12) Patent Application Publication
(10) Pub. No.: US 2014/0059721 A1
(43) Pub. Date: Feb. 27, 2014

(54) RICE RESISTANT/TOLERANT TO HPPD INHIBITING HERBICIDES

(71) Applicant: Ricetec Aktiengesellschaft, Vaduz (LI)

(72) Inventors: Melissa Hinga, League City, TX (US); Melissa Shannon Moon, Pearland, TX (US); Venu Reddyvari Channarayappa, Friendswood, TX (US); Russell D. Rasmussen, League City, TX (US); Federico Cuevas, League City, TX (US)

(73) Assignee: Ricetec Aktiengesellschaft, Vaduz (LI)

(21) Appl. No.: 13/975,034

(22) Filed: Aug. 23, 2013

Related U.S. Application Data
(60) Provisional application No. 61/692,861, filed on Aug. 24, 2012.

(51) Int. Cl. C12N 15/82

(52) U.S. Cl. CPC C12N 15/8274
USPC 800/300; 504/348; 504/271; 536/23.6; 435/6.11

(57) ABSTRACT

A mutant rice resistant/tolerant to 4-hydroxyphenylpyruvate dioxygenase (HPPD) inhibiting herbicides is disclosed. The HPPD herbicides useful for selection and weed control include mesotrione, benzobicyclon, and combinations thereof. This rice is useful for weed control. Molecular markers useful for selection are also disclosed.

This application claims priority from pending U.S. Provisional application 61/692,861 filed Aug. 24, 2012.

### SEQUENCE LISTING

The instant application contains a Sequence Listing which has been submitted in ASCII format via EFS-Web and is hereby incorporated by reference in its entirety. Said ASCII copy, created on Aug. 22, 2013, is named 701850_ST25.txt and is 9,790 bytes in size.

### BACKGROUND

Mutant rice is disclosed that is resistant/tolerant to 4-hydroxyphenylpyruvate dioxygenase (HPPD) inhibiting herbicides. Methods to produce the mutant rice plants, selecting and genotyping mutant rice, and methods to use mutant rice crops for weed control, are presented.

### Value of Rice Crops

Rice is an ancient agricultural crop and today is one of the principal food crops of the world. There are two cultivated species of rice: Oryza sativa L., the Asian rice, and Oryza glaberrima Steud., the African rice. The Asian species constitutes virtually all of the worlds cultivated rice and is the species grown in the United States. Three major rice producing regions exist in the United States: the Mississippi Delta (Arkansas, Mississippi, northeast Louisiana, southeast Missouri), the Gulf Coast (southwest Louisiana, southeast Texas), and the Central Valley of California.

Rice is one of the few crops that can be grown in a shallow flood as it has a unique structure allowing gas exchange through the stems between the roots and the atmosphere. Growth in a shallow flood results in the best yields and is the reason that rice is usually grown in heavy clay soils or soils with an impermeable hard pan layer just below the soil surface. These soil types are usually either not suitable for other crops or at best, the crops yield poorly.

The constant improvement of rice is imperative to provide necessary nutrition for a growing world population. A large portion of the world population consumes rice as their primary source of nutrition and crops must thrive in various environmental conditions including competing with weeds and attacks by unfavorable agents. Rice improvement is carried out through conventional breeding practices and by recombinant genetic techniques. Though appearing straightforward to those outside this discipline, crop improvement requires keen scientific and artistic skill and results are generally unpredictable.

Although specific breeding objectives vary somewhat in the different rice producing regions, increasing yield is a primary objective in all programs.

Plant breeding begins with the analysis and definition of strengths and weaknesses of cultivars in existence, followed by the establishment of program goals, to improve areas of weakness to produce new cultivars. Specific breeding objectives include combining in a single cultivar an improved combination of desirable traits from the parental sources. Desirable traits may be introduced due to spontaneous or induced mutations. Desirable traits include higher yield, resistance/tolerance to environmental stress, diseases and insects, better stems and roots, tolerance to low temperatures, better agronomic characteristics, nutritional value and grain quality.

For example, the breeder initially selects and crosses two or more parental lines, followed by selection for desired traits among the many new genetic combinations not previously combined in nature. The breeder can theoretically generate billions of new and different genetic combinations via crossing. Breeding by using crossing and selfing, does not imply direct control at the cellular level. However, that type of control may be achieved in part using recombinant genetic techniques.

Pedigree breeding is used for the improvement of self-pollinating crops such as rice. For example, two parents which possess favorable, complementary traits are crossed to produce an F₁ generation. One or both parents may themselves represent an F₁ from a previous cross. Subsequently a segregating population is produced, by growing the seeds resulting from selfing one or several F₁s if the two parents are pure lines, or by directly growing the seed resulting from the initial cross if at least one of the parents is an F₁. Selection of the best individual genomes may begin in the first segregating population or F₂; then, beginning in the F₃, the best individuals in the best families are selected. “Best” is defined according to the goals of a particular breeding program e.g., to increase yield, resist diseases. Overall a multifactorial approach is used to define “best” because of genetic interactions. A desirable gene in one genetic background may differ in a different genetic background. In addition, introduction of the gene may disrupt other favorable genetic characteristics previously in the rice, that is, have a different effect (phenotype). Replicated testing of families can begin in the F₄ generation to improve the effectiveness of selection for traits with low heritability. At an advanced stage of inbreeding (i.e., F₆ and F₇), the best lines or mixtures of phenotypically similar lines are tested for potential commercial release as new parental lines.

Backcross breeding is used to transfer genes for a highly heritable trait into a desirable homozygous cultivar or inbred line which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The process is used to recover all of the beneficial characteristics of the recurrent parent with the addition of the new trait provided by the donor parent.

Promising advanced breeding lines are thoroughly tested and compared to appropriate standards in environments representative of the commercial target area(s) for at least three or more years. The best lines are candidates for new commercial varieties or parents of hybrids; those still deficient in a few traits may be used as parents to produce new populations for further selection.

These processes, which lead to the final step of marketing and distribution, usually take from 8 to 12 years from the time the first cross is made and may rely on the development of improved breeding lines as precursors. Therefore, development of new cultivars is not only a time-consuming process, but requires precise forward planning, efficient use of resources, and a minimum of changes in direction. The results include novel genetic combinations not found in nature.

Some improvement of rice through breeding may be restricted to new combination of the natural genetic variation in rice and hybridizing species, such as wild rice. The introduction of new variation in a breeding program is usually through the crossing program as described, such as pedigree or backcross breeding. However, occasionally natural mutations are found that result in the introduction of new traits such as disease resistance/tolerance or height changes. Breeders have also developed new traits by inducing mutations (small changes in the DNA sequence) into a rice genome. Some of these mutations or combination of genes are not found in nature. Commonly, EMS or sodium azide plus MNU are used as mutagenic agents. These chemicals randomly induce single base changes in DNA, usually of G and C changed to A and T. Overall effects are unpredictable. Most of these changes have no effect on the crop as they fall either outside the gene coding regions or don't change the amino acid sequence of the gene product. However, some produce new traits or incorporate new DNA changes into previously existing lines.

The breeder has no direct control of mutation sites in the DNA sequence. The identification of useful changes is due to the random possibility that an effective mutation will be induced and that the breeder will recognize the phenotypic effects of the change and will be able to select rice having that mutation and introgress it into favorable genetic backgrounds. Seeds are treated with the mutagenic chemical and immediately planted to grow and produce M2 seed. The M2 seed will carry numerous new variations; therefore, no two experiments will produce the same combinations. Among these variations new traits previously not existing in rice and unavailable for selection by a plant breeder, may be found and used for rice improvement.

To find new traits the breeder must use efficient and strategic selection strategies as the process is completely random and has an extremely low frequency of useful new combinations. Among thousands of induced new genetic variants there may be only one with a desirable new trait. An optimal selection system will screen through thousands of new variants and allow detection of a few or even a single plant that might carry a new trait. After identifying or finding a possible new trait the breeder must develop a new cultivar by pedigree or backcross breeding and extensive testing to verify the new trait and cultivar exhibits stable and heritable value to rice producers.

Using recombinant genetic techniques, nucleic acid molecules with mutations that encode improved characteristics in rice, may be introduced into rice with commercially suitable genomes. After a mutation is identified by whatever course, it may be transferred into rice by recombinant techniques.

### Applications of Herbicide Resistance/Tolerance Patents in Rice

Weeds and other competitors for resources in crop fields compete for resources and greatly reduce the yield and quality of the crop. Weeds have been controlled in crops through the application of selective herbicides that kill the weeds but do not harm the crop. Usually selectivity of the herbicides is based on biochemical variations or differences between the crop and the weeds. Some herbicides are non-selective, meaning they kill all or almost all plants. Non-selective or broad spectrum herbicides can be used in crops if new genes are inserted that express specific proteins that convey tolerance or resistance/tolerance to the herbicide. Resistance/tolerance to herbicides has been achieved in crops through genetic mutations that alter proteins and biochemical processes. These mutations may arise in nature, but mostly they have been induced in crops or in vitro in tissue cultures or by inducing mutations in vivo. Unfortunately in some instances, especially with repeated use of a particular herbicide, weeds have developed resistance/tolerance through the unintended selection of natural mutations that provide resistance/tolerance. When weeds become resistant/tolerant to a particular herbicide, that herbicide is no longer useful for weed control. The development of resistance/tolerance in weeds is best delayed through alternating the use of different modes of action to control weeds, interrupting development of resistant/tolerant weeds.

Rice production is plagued by broad leaf plants and a particularly hard to control weed called red rice. One difficulty arises because red rice is so genetically similar to cultivated rice (they occasionally cross pollinate) that there are no selective herbicides available that target red rice, yet do not harm the cultivated rice. Control is currently provided in commercial rice production through the development of mutations found in rice that render rice resistant/tolerant to broad spectrum herbicides e.g. imidazolinone and sulfonylurea herbicides. Rice resistant/tolerant to herbicides that inhibit other deleterious plants, such as broad leaf plants, are needed.

Finding new mutations in rice that makes it resistant/tolerant to a variety of herbicides, and to combinations of herbicides with alternative modes of action, would greatly benefit rice production. Obtaining and incorporating genes for herbicide resistance/tolerance into rice genomes with additional favorable characteristics and alternative resistance/tolerances is challenging, unpredictable, time consuming and expensive, but necessary to meet the world's increasing food needs.

### SUMMARY

Described and disclosed herein are novel and distinctive rice lines with unique resistance/tolerances to herbicides in particular HPPD inhibiting herbicides and combinations thereof. For example, a mutant rice line designated ML0831266-03093 (ATTC Accession Number PTA-13620) is disclosed that is resistant/tolerant to HPPD inhibiting herbicides. The HPPD inhibiting herbicides include mesotrione, benzobicyclon, and combinations thereof.

Detection of genetic differences among rice plants explored quantitative trait loci (QTL) for tolerance or sensitivity to HPPD-inhibitor herbicides, such as mesotrione herbicides. Rice plants possessing these QTLs map to two chromosomal regions. Genetic markers are disclosed that are indicative of phenotypes associated with tolerance, improved tolerance, or susceptibility to HPPD inhibiting herbicides. Methods of introgressing such tolerance into a plant by breeding, transgenically or by a combination thereof, are disclosed. Plant cells, plants, and seeds tolerant to HPPD inhibiting herbicides are also provided.

A method to control weeds in a rice field, wherein the rice in the field includes plants resistant/tolerant to HPPD inhibiting herbicides, includes:
a. using herbicide resistant/tolerant rice in the rice field; and
b. contacting the rice field with an effective amount of HPPD inhibiting herbicide.

These rice lines should extend the useful life of several herbicides due to being able to rotate the kinds of herbicides applied in grower's fields thus slowing the development of weed resistance/tolerance. Several methods are possible to deploy these resistance/tolerances into hybrids or varieties for weed control, as well as options for hybrid seed production. The rice lines described herein represent new methods for weed control in rice and can be deployed in any of many possible strategies to control weeds and provide for long-term use of this and other weed control methods. In particular, mutant rice tolerant to HPPD inhibiting herbicides is disclosed.

Rice production for good yields requires specific weed control practices. Some herbicides are applied at the time of planting and others are applied before a permanent flood is applied, few weeds can grow in a full flood.

Through developing sources of resistance/tolerance to multiple herbicides, more options are available for weed control in rice. The rice line claimed should provide the ability to use herbicides with a new mode of action for weed control. The ability to use an HPPD inhibiting herbicide represents a mode of action not previously reported in rice. The use of these rice lines including combining lines with resistance/tolerance to herbicide with other modes of action provides new options for weed control in grower's fields thus slowing the development of weed resistance/tolerance. Several methods are disclosed to deploy this resistance/tolerance in hybrids for weed control as well as options for hybrid seed production.

Cells derived from herbicide resistant/tolerant seeds, plants grown from such seeds and cells derived from such plants, progeny of plants grown from such seed and cells derived from such progeny are within the scope of this disclosure. The growth of plants produced from deposited seeds, and progeny of such plants will typically be resistant/tolerant to HPPD inhibiting herbicides at effective levels of herbicides, that is, levels that would normally inhibit the growth of a corresponding wild-type plant.

A method for controlling growth of weeds in the vicinity of rice plants is also within the scope of the disclosure. One example of such methods is applying one or more herbicides to the fields of rice plants at levels of herbicide that would normally inhibit the growth of a rice plant. For example, at least one herbicide inhibits HPPD activity. Such methods may be practiced with any herbicide that inhibits HPPD activity and any resistant/tolerant rice mutation, e.g., embodiments disclosed herein.

A method for growing herbicide-tolerant rice plants includes (a) planting resistant/tolerant rice seeds; (b) allowing the rice seeds to sprout; (c) applying one or more herbicides to the rice sprouts at levels of herbicide that would normally inhibit the growth of a rice plant. For example, at least one of the herbicides inhibits HPPD. Such methods may be practiced with any herbicide that inhibits HPPD activity.

Methods of producing herbicide-tolerant rice plants may also use a transgene. One example of such a method is transforming a cell of a rice plant with a transgene, wherein the transgene encodes an HPPD enzyme that confers tolerance in resulting rice plant to at least one herbicide. Any suitable cell may be used in the practice of these methods, for example, the cell may be in the form of a callus.

### BRIEF DESCRIPTION OF DRAWINGS

FIG. 1A: Results from an initial experiment on the mutant line (ML0831266-03093) showing results of three herbicide treatments with mesotrione. The first two treatments were applied immediately following planting. The third treatment included a herbicide application immediately following planting followed by a foliar application at the three leaf state.

FIG. 1B: Rate response of the mutant line ML0831266-03093, the unmutated original line P1003, and a different type of rice R0146. Mesotrione was applied across all plots immediately following planting at a rate of 210 gai/ha. The response rates were applied at the 2-3 leaf stage. Response was recorded twenty-one days following the foliar application.

FIG. 2: Picture of controls used in the mesotrione herbicide bioassay after spraying one application of mesotrione at 105 gai/ha followed by a second application of 630 gai/ha (A). The mutant line ML0831266-03093 shows little damage from the spray application, (B); while the unmutated type P1003 is severely damaged and a different type of rice, (C) R0146 with sensitivity to mesotrione is completely killed.

FIG. 3: Response of different types of rice to mesotrione applied at the 2-3 leaf stage; wherein different rates of mesotrione are applied.

FIG. 4: QTL mapping results; (A) population sprayed with mesotrione at 105 gai/ha; (B) polulation sprayed with mesotrione at 420 gai/ha; (C) population sprayed with mesotrione at 105 gai/ha followed by a second application at 630 aig/ha; R0146 is sensitive to mesotrione. Each population was phenotyped with a different herbicide bioassay using mesotrione as indicated on the three chromatograms.

### DETAILED DESCRIPTION

### Mutation Population and Establishment

A mutation breeding program was initiated to develop proprietary herbicide tolerant lines. A permanent mutant population was created by exposing approximately 10,000 seeds (estimated by the average weight of a kernel) of three rice lines including P1003, R0146, and P1062 to mutagens sodium azide (AZ) and methyl-nitrosourea (MNU). The treated seeds were space planted. Individual plants were harvested creating 8,281 mutation lines. The mutation lines have been maintained as a permanent mutant population for trait screening.

### Herbicide Screening

Mesotrione (Callisto®), is an herbicide that inhibits the plant enzyme 4-hydroxyphenylpyruvate dioxygenase (HPPD). Callisto® is a postemergent and preemergent herbicide used to control annual broadleaf weeds in corn and certain other crops. The herbicide only damages some rice at lower rates but kills other types of rice. All rice appears to be at least damaged by higher herbicide rates. Finding resistance/tolerance to Callisto® herbicide in rice results in a new mode of action for controlling broadleaf weeds in rice.

Resistance/tolerance to mesotrione herbicide was found by screening the permanent mutant population in Alvin, Tex. All lines in the permanent mutant population were planted into a dry seed bed. Within twelve hours after planting mesotrione (Callisto®) was applied at a rate of 255.1 gm ai per acre. The field was immediately flushed with water and kept moist through periodic flushing. The seedlings grew, many were bleached white and all lines derived from R0146 died whereas plants lived from 21 lines derived from the P1003 and 2 derived from P1062 mutation populations.

### Resistant/Tolerant Plants

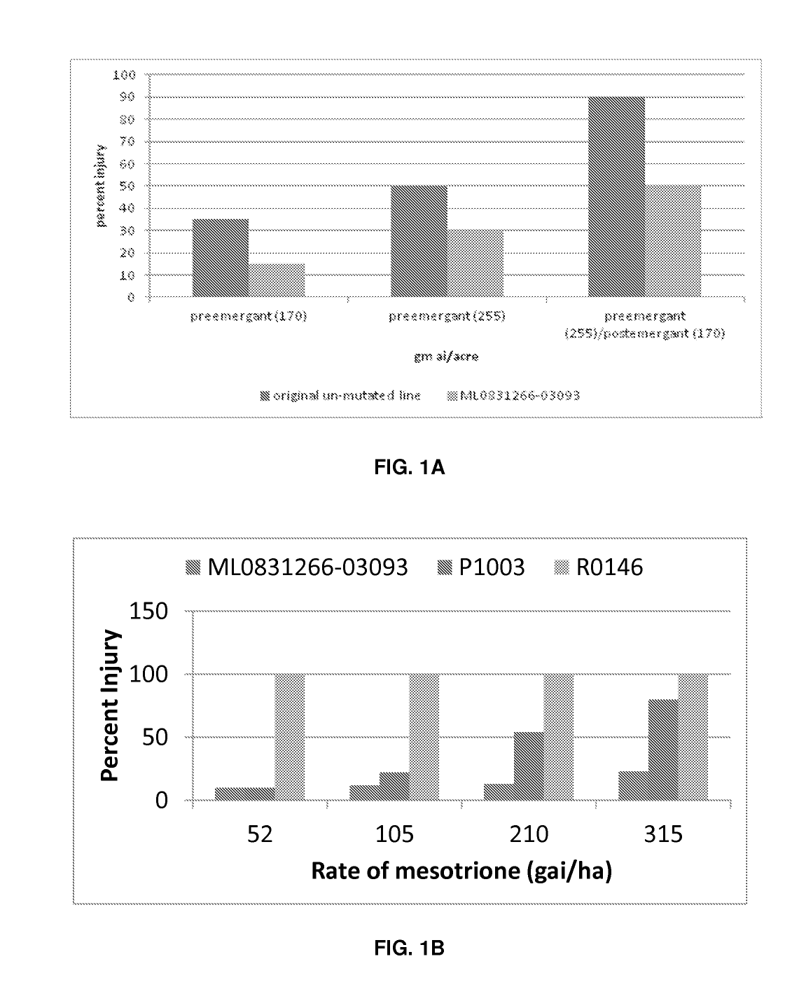

After the initial screening of the mutation population, the lines that had less damage were selected and tested in additional experiments using different rates of the herbicide. In particular a rate response experiment was conducted in which two different rates of mesotrione were applied pre-emergence, plus an additional foliar application was also applied. This experiment differentiated one mutant line as having superior resistance/tolerance (less injury) to the mesotrione herbicide as compared to the un-mutated control (FIG. 1, Table 1). Progeny of this mutant line designated ML0831266-03093 are maintained as a new line carrying resistance/tolerance to mesotrione herbicide. Seeds are deposited under ATCC PTA-13620. The line (ML0831266-03093) is a source of resistance/tolerance that is backcrossed into proprietary rice lines or used directly in breeding to develop new proprietary rice lines. The developed lines are a source of herbicide resistance/tolerance for use in development of new hybrids that offer an alternative mode of action to control weeds in rice. Affording this opportunity to growers is of great value both in providing high yields and in extending the useful life of available weed control technologies. These herbicide resistant/tolerant traits can be tracked through the simple application of herbicides to growing plants.

### Causal Mutation for Resistance

Genetic mapping for the genomic location for resistance/tolerance in line ML0831266-03093 is carried out by QTL mapping strategies known in the art. (See Materials and Methods) The resistant/tolerant line ML0831266-03093 may be crossed with a highly sensitive line and the resulting F1 seeds grown and plants selfed to produce a F2 mapping population segregating for the resistance/tolerance trait. Finding trait linked markers is done by genotyping each F2 plant, spraying the plants with an appropriate (effective) concentration of the herbicide, and associating the molecular genotypes with the phenotype of each individual. This process identifies a genomic region between markers for the causal mutation for resistance/tolerance.

The entire genomic region identified through genetic mapping is then sequenced for the mutant line and the original non-mutant line to identify DNA sequence changes in the mutant line. The sequence differences are then further characterized and validated through testing the sequencing differences in large phenotyped segregating populations.

In another method next generation sequencing is used to sequence and compare a bulk of individuals that are resistant/tolerant to either a bulk of susceptible individuals or to the original non-mutant line. In this method the causal mutation is found through the resistant/tolerant bulk having the highest portion of the mutation or sequence difference as compared to the susceptible bulk or non-mutant original line. Within the bulk the sequence reads for the causal mutation or sequence change should be all or very close to all homozygous for the mutation. Other mutations or sequence changes will have varying portions of the reads with either the wild-type sequence or the mutation sequence. In this manner ideally a single mutation can be predicted as the causal mutation or less ideally a small set of mutations may be the probable cause for the phenotype. These suspect mutations may be characterized and validated as with sequencing by making a target site markers and testing them on large phenotyped segregating populations.

### ATCC Deposit

A deposit of the RiceTec, Inc. ML0831266-0393 disclosed above and recited in the appended claims has been made with the American Type Culture Collection (ATCC), 10801 University Boulevard, Manassas, Va. 20110. The date of deposit was Mar. 19, 2013. All restrictions will be removed upon granting of a patent, and the deposit is intended to meet all of the requirements of 37 C.F.R. §§1.801-1.809. The ATCC Accession Number is PTA-13620. The deposit will be maintained in the depository for a period of thirty years, or five years after the last request, or for the enforceable life of the patent, whichever is longer, and will be replaced as necessary during that period.

### EXAMPLES

### Example 1

### Production of Hybrid Rice Resistant/Tolerant to One HPPD Inhibiting Herbicide

The HPPD inhibiting herbicide resistance/tolerance provided by ML0831266-03093 is deployed individually into hybrids through either the male or female parent resulting in the hybrid seed being resistant/tolerant to the herbicide. If the resistance/tolerance is deployed in only the male parent, then in addition to its use for weed control, the herbicide when applied to hybrid seed kills contaminating female selfed seed. On the other hand if the resistance/tolerance is deployed only through the female parent, growers may eliminate contaminating male selfed seed.

Growers may alternate the type of resistance/tolerance they purchase, and apply in their fields to reduce the chance that weeds develop resistance/tolerance to the herbicide. The herbicide though primarily for control of broad leaf weeds also allows for some enhanced control of red rice as at higher rates it will kill certain types of rice. If resistance/tolerance arose in red rice from cross pollination, it could still be controlled with the other herbicide class in the next season.

### Example 2

### Production of Hybrid Rice with High Level of Resistant/Tolerant to HPPD Inhibiting Herbicides

The HPPD inhibiting herbicide resistance/tolerance provided by ML0831266-03093 is deployed into both the male and female parents of a hybrid. The resulting hybrid seed will carry resistance/tolerance to mesotrione and other HPPD inhibiting herbicides. Resistance/tolerance provided in this manner is stronger and offers better weed control through the possibility of being able to apply higher rates of herbicide.

### Example 3

### Production of Hybrid Rice Resistant/Tolerant to Multiple Herbicides

The herbicide resistance/tolerance for at least 2 herbicides is deployed in a single hybrid through making both the female and male parent resistant/tolerant to both herbicides. Deployment in this manner results in hybrid seed being homozygous for both resistance/tolerances. By providing resistance/tolerance in homozygous condition in the hybrid for both herbicide classes the hybrid seed shows maximum level of resistance/tolerance. In addition by deploying both resistance/tolerances together, the grower has the option to select either herbicide to apply in a given season, alternatively, both herbicides could be applied within the same season. The ability to rotate herbicides provides the opportunity to extend the life of the herbicides through delaying the development of weed resistance/tolerance. This method also allows for the use of both herbicide classes for weed control during hybrid seed production.

### Example 4

### Production of Hybrid Rice Resistant/Tolerant to Mesotrione and at Least One Other Herbicide Class

1. Resistance/tolerance to mesotrione and at least one other herbicide class is deployed in a single hybrid by using a male parent that carries resistance/tolerance to the mesotrione (or the other herbicide) and a female that carries the other resistance/tolerance. The method allows the grower to make a single purchase but to be able to choose which herbicide to apply. A single class of herbicide may be used in any one season and rotated between seasons, or alternatively both herbicides could be applied within a single season. In addition deployment by this method, contaminating selfed seed of both parents is eliminated in the hybrid seed through application of both herbicides, or one type or the other, eliminated through application of only one herbicide.

2. In another method of deployment the mesotrione resistance/tolerance and any other herbicide resistance/tolerance could be deployed through making a hybrid with a male parent that carries both resistance/tolerances. The grower then has the option to choose which herbicide class to apply or to apply both within a single season. In addition, through the application of either herbicide contaminating selfed female seed would be eliminated. Alternatively both herbicide class resistance/tolerances are provided in the female parent, giving the grower the same options for weed control.

3. Another embodiment is to deploy the mesotrione resistance/tolerance to both parents, and another herbicide resistance/tolerance into only one parent, such as the male parent. The hybrid seed are then homozygous for the mesotrione resistance/tolerance but not the other. A scheme like this is used to make an early application with the herbicide put into only the male parent, providing weed control and elimination of contaminating female selfs. Later in the season mesotrione may be applied or another HPPD inhibitor herbicide. The useful life of both herbicides is extended through limiting or eliminating the development of weed resistance/tolerance. In another application this method allows the use of mesotrione or other HPPD inhibitor herbicide to control weeds in seed production fields, allowing for cleaner seed.

4. Alternatively a different herbicide could be deployed in both of the hybrid parents and the mesotrione/HPPD inhibitor is deployed in only the male parent.

5. Other embodiments for deploying herbicide resistant/tolerant lines include other traits such as resistance/tolerance to other classes of herbicides, or other traits of importance.

### Example 5

### Seed Production

The herbicide resistance/tolerance is also used for seed production. As an example, if it is deployed into the female parent, making it resistant/tolerant, the herbicide is applied to the seed production field to kill the male plants before setting seed so that a seed production field is harvested as a bulk. In addition the purity of the seed may also be verified through deploying two herbicide resistance/tolerances with only one in each parent. Selfed seed is detected and eliminated by applying herbicide put into the other parent.

### Example 6

### Control of Broadleaf Weeds and Limited Control of Grasses

The resistance/tolerance when deployed in a hybrid, by any combination, provides resistance/tolerance to mesotrione or other HPPD inhibiting herbicides. This deployment results in a new mode of action in rice to control broadleaf weeds with some limited control of grasses such as red rice.

### Example 7

### Selection of Herbicide Resistant/Tolerant Rice Using a Herbicide Bioassay

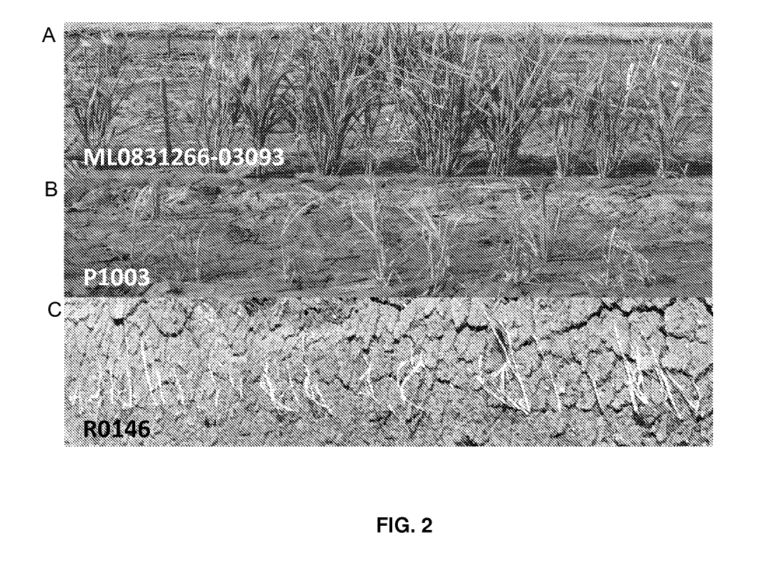

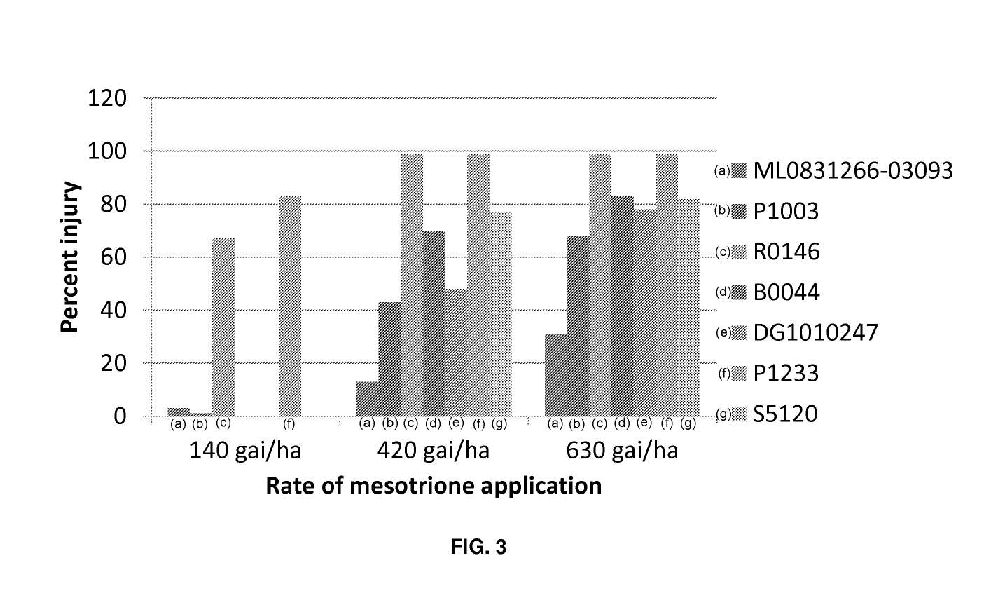

Selection of material inheriting mesotrione tolerance is accomplished by a simple herbicide bioassay. A high rate of mesotrione (at least 420 gm ai/ha) is applied allowing differentiation of heterozygous individuals from homozygous individuals and the tolerance level of the mutation line from the inherent tolerance level in the background of some types of rice. In one example a rate of 105 gm ai/ha is applied followed three weeks later by a second application of 630 gm ai/ha. In another example a rate of 420 gm ai/ha is applied in a single application. Yet another example entails applying the herbicide at a rate of 630 gm ai/ha. Herbicide applications are done at the three to four leaf stage of seedling growth. The ideal situation is to have also planted near or within the plants to be selected a set of plants from the original mutant mesotrione donor line ML0831266-03093, a row of plants of the wild-type of the mutation line, P1003, and a row of the line involved as the other parent in the cross. These control lines allow easy differentiation for inheritance of the tolerance provided by the ML0831266-03093 mutant line through comparison of the response in the plants to be selected with the control lines. Only plants that live and are healthy will have inherited and be homozygous for the tolerance level provided by the ML0831266-03093 mutant line (FIG. 2).

### Example 8

### Mutant Rice ML0831266-03093

The mutant line ML0831266-03093 was demonstrated to carry a high tolerance level to mesotrione herbicide beyond the tolerance found naturally in some rice types including the original mutation treated line P1003. The mutant line was planted in rows or alternatively whole plots were planted and rows of the unmutated line (P1003) and other types of rice or whole plots were planted. Mesotrione was applied pre-emergence or alternatively it was applied post-emergence at the three to four leaf stage of the rice plants. Various rates of mesotrione were applied pre-emergence, pre-emergence followed by post-emergent, or post-emergent with a single or sequential application. Post-emergent applications were applied at the 3-4 leaf stage of the rice.

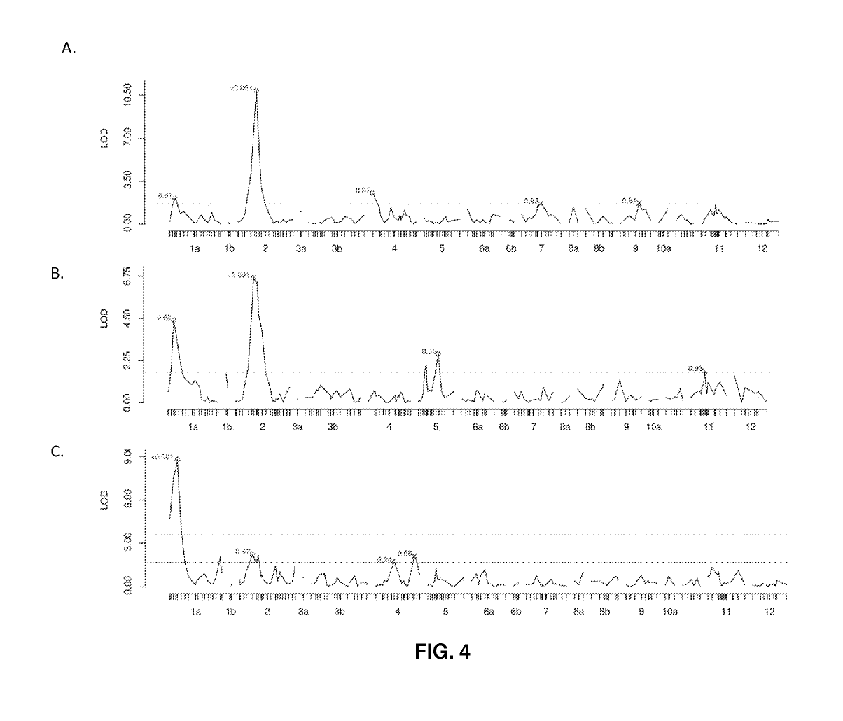

With low rates (105 gm ai/ha) of mesotrione applied, both the mutant line as well as the original unmutated line survived. However, other types of rice, such as line R0146 were killed at these rates of mesotrione. Applying mesotrione herbicide at higher rates clearly shows new and novel tolerance level as only the mutant line ML0831266-03093 survives while the original unmutated line P1003 and all other tested lines are killed or severally injured. The higher tolerance to mesotrione makes the line ML0831266-03093 of commercial value as both the tolerance can be controlled or bred into new varieties and it is of a high enough level to allow commercial weed control in rice with the application of mesotrione herbicide and possibly other HPPD inhibiting herbicides [FIG. 1(A) initial results, FIG. 1(B) new results after 1 year, and FIG. 4-trial results on various rice lines].

### Example 9

### Chromosomal Locations of Mutations Related to HPPD Inhibiting Resistance/Tolerance

The mesotrione tolerant line ML0831266-03093 was tolerant to rates of 420 gm ai/ha and even a dual application of mesotrione first at a rate of 105 gm ai/ha followed three weeks later by an application of 630 gm ai/ha. The line ML0831266-03093 with this high level of tolerance was crossed to the highly sensitive line R0146. The cross was made and the resulting F1 seeds were harvested, planted, and allowed to self to produce a F2 population. The F2 population was grown and tissue was collected from individual plants. Each F2 plant and parental lines was tested with a set of approximately 195 evenly spaced molecular markers that are polymorphic between the parent lines ML0831266-03093 and R0146 (see Materials and Methods).

Seedlings of size 3 to 4 leaves were sprayed with mesotrione herbicide. In one set of 89 plants mesotrione was first applied at 105 gm ai/ha killing 23 plants. Both the mutant line control ML0831266-03093 and the unmutated line P1003 survived the herbicide application while the unmutated line R0146 was killed. The surviving plants including some additional plants making a total of 95 which were then sprayed with another treatment of mesotrione at a rate of 630 gm ai/ha killing or injuring 67 while 28 survived. The surviving ratio of plants to those killed or injured fits a one quarter ratio with a chi squared value of 1.03, well within the expected for a single recessive gene. Another set of F2 individuals of size 78 individuals were sprayed with a single application of mesotrione herbicide at a rate of 420 gm ai/ha resulting in 26 plants surviving also fitting in the expected one quarter ratio for a single recessive gene with a chi square value of 2.88 (Table 4).

Genetic mapping of the three groups of F2 individuals including the set of individuals sprayed with mesotrione at only 105 gm ai/ha, the set followed by a sequential application of 630 gm ai/ha, and the final group sprayed with 420 gm ai/ha shows two genes controlling resistance/tolerance to mesotrione. In the population sprayed with 105 gm ai/ha a single QTL found on chromosome 2 with linkage to SNP marker BG-id2004662 acted in a mostly dominant manner. This marker and QTL is linked to the inherent tolerance in line P1003. The marker is useful for breeding and selection of new mesotrione tolerant lines. The discovery of this QTL facilitates commercial development of new rice varieties with a new method for controlling weeds through the use of mesotrione herbicide.

In the two groups of F2 individuals sprayed with the higher rates of mesotrione (420 and 630 gm ai/ha) a second QTL was found with linkage to SNP marker WG-id1002788. This QTL is linked to the probable genetic position of the causal mutation for high tolerance to mesotrione. The combined tolerance of the QTL developed through mutation breeding on chromosome 1 and the QTL discovered in line P1003 provides a novel tolerance to mesotrione and combined with the linked molecular markers facilitates quick and efficient breeding of new rice varieties (FIG. 4).

### Example 10

### Identification of the Causal Mutation for Tolerance to Mesotrione and Other HPPD Herbicides

The mutant line ML0831266-03093 is crossed back to the original line, P1003, used for induction of mutations. The F1 seed is grown and the plants selfed to produce an F2 population segregating for tolerance to mesotrione. Each F2 plant is labeled and a leaf sample is collected for DNA extraction. The F2 plants are then sprayed with mesotrione herbicide at the 3-4 leaf stage at a rate of 630 gmai/ha. The plants resembling the mutant line ML0831266-03093 control should be homozygous for the causal mutation and among which a set of 20 is selected for bulking DNA to make a sample for sequencing. Out of the plants killed by the herbicide application a set of twenty is also identified for DNA extraction to represent individuals that have the wild-type non-mutant allele at the causal tolerance locus.

The leaf tissue from the identified individuals is used for DNA extraction and DNA is combined from each set to make a bulk of individuals carrying the tolerance and a bulk lacking the tolerance. In addition DNA is also extracted from leaf tissue derived from the original line, P1003, used for mutation treatment. These samples are sequenced at 30× coverage and compared to the rice reference sequence, Nipponbare, to the original line used for mutation treatment, P1003, and to each other. Within these comparisons with the tolerant bulk the causal mutation should be homozygous for the mutant type in all or nearly all sequencing reads. Molecular markers are then designed for the suspected causal mutations a tested on a larger phenotyped segregating population to validate the suspected causal mutations.

Other characterizations and processes are also applicable to verify the function of the causal mutation. For example the gene containing the mutation is identified through comparison to the published full rice sequence and related databases. Furthermore the gene product or enzyme can be isolated and characterized to describe its normal function and function with the new mutation especially in relation to its function in the presence of mesotrione.

### DEFINITIONS

In the description and tables which follow, a number of terms are used. In order to provide a clear and consistent understanding of the specification and claims, including the scope to be given such terms, the following definitions are provided:

Allele. Allele is any one of many alternative forms of a gene, all of which generally relate to one trait or characteristic. In a diploid cell or organism, the two alleles of a given gene occupy corresponding loci on a pair of homologous chromosomes.

Backcrossing. Process of crossing a hybrid progeny to one of the parents, for example, a first generation hybrid F1 with one of the parental genotypes of the F1 hybrid.

Blend. Physically mixing rice seeds of a rice hybrid with seeds of one, two, three, four or more of another rice hybrid, rice variety or rice inbred to produce a crop containing the characteristics of all of the rice seeds and plants in this blend.

Cell. Cell as used herein includes a plant cell, whether isolated, in tissue culture or incorporated in a plant or plant part.

Cultivar. Variety or strain persisting under cultivation.

Embryo. The embryo is the small plant contained within a mature seed.

Essentially all the physiological and morphological characteristics. A plant having essentially all the physiological and morphological characteristics of the hybrid or cultivar, except for the characteristics derived from the converted gene.

Genetic map. Is a description of genetic linkage relationships among loci on one or more chromosomes (or linkage groups) within a given species, generally depicted in a diagrammatic or tabular form.

Germplasm. Means the genetic material that comprises the physical foundation of the hereditary qualities of an organism. As used herein, germplasm includes seeds and living tissue from which new plants may be grown; or, another plant part, such as leaf, stem, pollen, or cells, that may be cultured into a whole plant. Germplasm resources provide sources of genetic traits used by plant breeders to improve commercial cultivars.

Grain Yield. Weight of grain harvested from a given area. Grain yield could also be determined indirectly by multiplying the number of panicles per area, by the number of grains per panicle, and by grain weight.

Introgression. Refers to the transmission of a desired allele of a genetic locus from one genetic background to another by sexual crossing, transgenic means, or any other means known in the art. For example, introgression of a desired allele at a specified locus can be transmitted to at least one progeny plant via a sexual cross between two parent plants, at least one of the parent plants having the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene or a gene allele that imparts resistance to a plant pathogen.

Linkage. Refers to a phenomenon wherein alleles on the same chromosome tend to segregate together more often than expected by chance if their transmission was independent. Genetic recombination occurs with an assumed random frequency over the entire genome. Genetic maps are constructed by measuring the frequency of recombination between pairs of traits or markers. The closer the traits or markers lie to each other on the chromosome, the lower the frequency of recombination, and the greater the degree of linkage. Traits or markers are considered herein to be linked if they generally co-segregate. A 1/100 probability of recombination per generation is defined as a map distance of 1.0 centiMorgan (1.0 cM).

Locus. A locus is a position on a chromosome occupied by a DNA sequence; it confers one or more traits such as, for example, male sterility, herbicide tolerance, insect resistance/tolerance, disease resistance/tolerance, waxy starch, modified fatty acid metabolism, modified phytic acid metabolism, modified carbohydrate metabolism and modified protein metabolism. The trait may be, for example, conferred by a naturally occurring gene introduced into the genome of the variety by backcrossing, a natural or induced mutation, or a transgene introduced through genetic transformation techniques. A locus may comprise one or more alleles integrated at a single chromosomal location.

Marker assisted selection. Refers to the process of selecting a desired trait or desired traits in a plant or plants by detecting one or more molecular markers from the plant, where the molecular marker is linked to the desired trait.

Mesotrione. Belongs to the triketone family of herbicides, which are chemically derived from a natural phytotoxin produced by the bottlebrush plant Callistemon citrinus. Mesotrione works by inhibiting HPPD (p-hydroxyphe-nylpyruvate dioxygenase), an essential enzyme in the biosynthesis of carotenoids. Carotenoids protect chlorophyll from excess light energy.

Plant. As used herein, the term “plant” includes reference to an immature or mature whole plant, including a plant from which seed or grain or anthers have been removed. Seed or embryo that will produce the plant is also considered to be the plant.

Plant Part. As used herein, the term “plant part” (or a rice plant, or a part thereof) includes protoplasts, leaves, stems, roots, root tips, anthers, seed, grain, embryo, pollen, ovules, cotyledon, hypocotyl, glumes, panicles, flower, shoot, tissue, cells, meristematic cells and the like.

Quantitative Trait Loci (QTL). Genetic loci that controls to some degree numerically measurable traits that are usually continuously distributed.

Regeneration. Regeneration refers to the development of a plant from tissue culture.

Single Gene Converted (Conversion). Single gene converted (conversion) includes plants developed by a plant breeding technique called backcrossing wherein essentially all of the desired morphological and physiological characteristics of an inbred are recovered, while retaining a single gene transferred into the inbred via crossing and backcrossing. The term can also refer to the introduction of a single gene through genetic engineering techniques known in the art.

Tolerance and improved tolerance. Are used interchangeably herein and refer to plants in which higher doses of an herbicide are required to produce effects similar to those seen in non-tolerant plants. Tolerant plants typically exhibit fewer necrotic, lytic, chlorotic, or other lesions when subjected to the herbicide at concentrations and rates typically employed by the agricultural community. A “tolerant plant” or “tolerant plant variety” need not possess absolute or complete tolerance such that no detrimental effect to the plant or plant variety is observed when the given herbicide is applied. Instead, a “tolerant plant,” “tolerant plant variety,” or a plant or plant variety with “improved tolerance” will simply be less affected by the given herbicide than a comparable susceptible plant or variety.

TABLE 1
Phenotypic characteristics of a mutant rice line showing resistance/tolerance
to mesotrione, with comparison to the original unmutated parent line.

| | Days to 50% | Plant | Plant | | Sheath | | | |
|---|---|---|---|---|---|---|---|---|
| Designation | Heading | Height | Type | Pubesence | Color | Awns | TKW, g | yield/plant |
| Unmutated | 85 | 96.5 | erect | glaborous | purple | None | 23.15 | N/A |
| parent line | | | | | inside sheath | | | |
| ML0831266- | 82 | 94 | erect | glaborous | purple | None | 24.55 | 4.5 |
| 03093F2 | | | | | inside sheath | | | |

TABLE 2
SNP markers

| | | | | | SEQ |
|---|---|---|---|---|---|
| | Chromo- | upstream | | downstream | ID |
| ID | some | sequence | Allele | sequence | NO: |
| WG-id1002788 | 1 | ATGCCCACGGCGGCGGC | C/T | GTCGGAAATGCCTGCCA | 4 |
| | | GGCGGAGGAGGAGGAG | | CGGGCTGTTCCCGCAGG | |
| | | GAGGAGGAGCTAAGGA | | TATTGAGAAATGAGCGC | |
| | | GCGGCGCGGTACGTCGC | | TGAGTTCCTGACGCGTTT | |
| | | CGGTGCTGTTCTGCTTTG | | AAATCCACTGATTAGCT | |
| | | TAGCCGCTGCTGTCCT | | GAGTTCCCTTCCAA | |
| WG-id1003490 | 1 | GAGAGTGGAGGAGGAG | A/G | CAAGCAAAGGAAGCAA | 5 |
| | | GACGAGTGGAGGTGGA | | GCAAAAGAAAAAAAGCC | |
| | | GGTGGCGCGCGGCTGC | | CGGGAATTTACCTGGCG | |
| | | GCGGTGCGCTTCTTTTTT | | GGAATGCCCTACTTGGC | |
| | | TTTTCTTTTTTTGTTCCCG | | AGCGCCGCCCGTCTCTCT | |
| | | CCGCAACCAAAGGAG | | CCACAAACGCCCTGC | |
| BG-id2004662 | 2 | TTCTATCTCAAGGCGGC | C/T | GCCAAATGTCTGATGAA | 6 |
| | | AATAGAATCATAGATGC | | TTGCTCTTGCTCTGATGT | |
| | | TAGAGTCCAGAAGAAGG | | TGAGCCCGATGAAGTTG | |
| | | CCAAAGACTTGAAATTTT | | TTAGCTGCTGAGGACAT | |
| | | CAGTTGAGAATGAGCAA | | GATCGGTACCACCTATAT | |
| | | TCCAAGGTGATGCT | | TGACAACCCTGAT | |
| WG-id2003988 | 2 | TCAGTGTTCACGGACCCT | A/G | TATCAATCGATCAATCAC | 7 |
| | | ACATGGAGTTCTCCTAA | | CAATCGGATGGTACCAA | |
| | | GTTCAACTACAAGAGAC | | ATCCAAAACAACAGTTG | |
| | | ATAGCCCATAGGGTAAT | | GGGAAAACTGATCCTAC | |
| | | GCCCTCACTTTCCAGCTC | | CAACCCAGCTCAACTAAT | |
| | | TTTAACTATGGAG | | TTTGCAGTGCTAC | |

TABLE 3
Markers used in QTL analysis

| | SEQ ID |
|---|---|
| MARKER | NO: |
| WG-id11001864 | 8 |
| WG-id11002275 | 9 |
| WG-id11003701 | 10 |
| WG-id11007323 | 11 |
| WG-wd10001341 | 12 |
| WG-wd11001701 | 13 |
| WG-wd2002275 | 14 |
| WG-wd7000143 | 15 |
| BG-id10001133 | 16 |
| BG-id10003147 | 17 |
| BG-id10004614 | 18 |
| BG-id1001716 | 19 |
| BG-id1012406 | 20 |
| BG-id1015060 | 21 |
| BG-id1020809 | 22 |
| BG-id1026723 | 23 |
| BG-id11000280 | 24 |
| BG-id11000643 | 25 |
| BG-id11001000 | 26 |
| BG-id11003263 | 27 |
| BG-id11005541 | 28 |
| BG-id11011578 | 29 |
| BG-id12001413 | 30 |
| BG-id12003453 | 31 |
| BG-id12006669 | 32 |
| BG-id12010130 | 33 |
| BG-id2000100 | 34 |
| BG-id2001406 | 35 |
| BG-id2002159 | 36 |
| BG-id2004662 | 37 |
| BG-id2006793 | 38 |
| BG-id2008132 | 39 |
| BG-id2009032 | 40 |
| BG-id2010498 | 41 |
| BG-id2012278 | 42 |
| BG-id2013398 | 43 |
| BG-id3002278 | 44 |
| BG-id3006415 | 45 |
| BG-id3007343 | 46 |
| BG-id3008063 | 47 |
| BG-id3008702 | 48 |
| BG-id3011050 | 49 |
| BG-id3011406 | 50 |
| BG-id3015075 | 51 |
| BG-id4001244 | 52 |
| BG-id4002084 | 53 |
| BG-id4004010 | 54 |
| BG-id4010543 | 55 |
| BG-id4012206 | 56 |
| BG-id5003430 | 57 |
| BG-id5004121 | 58 |
| BG-id5011704 | 59 |
| BG-id5014703 | 60 |
| BG-id6007975 | 61 |
| BG-id6011524 | 62 |
| BG-id6016683 | 63 |
| BG-id6016941 | 64 |
| BG-id7006069 | 65 |
| BG-id8000032 | 66 |
| BG-id8004971 | 67 |
| BG-id8006271 | 68 |
| BG-id9003596 | 69 |
| BG-ud11001609 | 70 |
| BG-ud7000168 | 71 |
| BG-ud7000468 | 72 |
| BG-ud7001467 | 73 |
| BG-ud9000404 | 74 |
| BG-ud9000939 | 75 |
| BG-wd12000096 | 76 |
| BG-wd5002107 | 77 |
| BG-wd7000537 | 78 |
| BG-wd8000300 | 79 |
| WG-id10000057 | 80 |
| WG-id1000027 | 81 |
| WG-id10000678 | 82 |
| WG-id10005716 | 83 |
| WG-id10006397 | 84 |
| WG-id10006890 | 85 |
| WG-id10007362 | 86 |
| WG-id1000987 | 87 |
| WG-id1002788 | 88 |
| WG-id1003490 | 89 |
| WG-id1004858 | 90 |
| WG-id1005915 | 91 |
| WG-id1006413 | 92 |
| WG-id1007758 | 93 |
| WG-id1008433 | 94 |
| WG-id1011077 | 95 |
| WG-id1013249 | 96 |
| WG-id1015747 | 97 |
| WG-id1019114 | 98 |
| WG-id1022207 | 99 |
| WG-id1023338 | 100 |
| WG-id12004473 | 101 |
| WG-id12005677 | 102 |
| WG-id12007189 | 103 |
| WG-id12008113 | 104 |
| WG-id12009381 | 105 |
| WG-id2000711 | 106 |
| WG-id2003035 | 107 |
| WG-id2003988 | 108 |
| WG-id2005453 | 109 |
| WG-id2005879 | 110 |
| WG-id2007502 | 111 |
| WG-id2011561 | 112 |
| WG-id2011986 | 113 |
| WG-id2014452 | 114 |
| WG-id2015344 | 115 |
| WG-id2016104 | 116 |
| WG-id3000020 | 117 |
| WG-id3003557 | 118 |
| WG-id3003855 | 119 |
| WG-id3004338 | 120 |
| WG-id3005216 | 121 |
| WG-id3005783 | 122 |
| WG-id3009997 | 123 |
| WG-id3010769 | 124 |
| WG-id3013945 | 125 |
| WG-id3016222 | 126 |
| WG-id3017628 | 127 |
| WG-id3018382 | 128 |
| WG-id4000023 | 129 |
| WG-id4001471 | 130 |
| WG-id4002895 | 131 |
| WG-id4004798 | 132 |
| WG-id4005527 | 133 |
| WG-id4005882 | 134 |
| WG-id4006725 | 135 |
| WG-id4007645 | 136 |
| WG-id4008100 | 137 |
| WG-id4008430 | 138 |
| WG-id4008947 | 139 |
| WG-id4009312 | 140 |
| WG-id4009705 | 141 |
| WG-id4011039 | 142 |
| WG-id4011619 | 143 |
| WG-id4011820 | 144 |
| WG-id5001055 | 145 |
| WG-id5002055 | 146 |
| WG-id5002453 | 147 |
| WG-id5002782 | 148 |
| WG-id5004697 | 149 |
| WG-id5006824 | 150 |
| WG-id5007247 | 151 |
| WG-id5007583 | 152 |
| WG-id5008807 | 153 |
| WG-id5009334 | 154 |
| WG-id5010535 | 155 |
| WG-id6000075 | 156 |
| WG-id6001960 | 157 |
| WG-id6002888 | 158 |
| WG-id6003335 | 159 |
| WG-id6004012 | 160 |
| WG-id6004657 | 161 |
| WG-id6005348 | 162 |
| WG-id6007016 | 163 |
| WG-id6010853 | 164 |
| WG-id6012703 | 165 |
| WG-id6014165 | 166 |
| WG-id6016119 | 167 |
| WG-id7000480 | 168 |
| WG-id7001929 | 169 |
| WG-id7002851 | 170 |
| WG-id7003936 | 171 |
| WG-id7004491 | 172 |
| WG-id8000555 | 173 |
| WG-id8001575 | 174 |
| WG-id8002235 | 175 |
| WG-id8005634 | 176 |
| WG-id8006703 | 177 |
| WG-id8007014 | 178 |
| WG-id8007344 | 179 |
| WG-id8007751 | 180 |
| WG-id9000056 | 181 |
| WG-id9002563 | 182 |
| WG-id9002755 | 183 |
| WG-id9005502 | 184 |
| WG-id9006187 | 185 |
| WG-id9006850 | 186 |
| WG-id9007344 | 187 |
| WG-ud1001267 | 188 |
| WG-ud7000348 | 189 |
| WG-ud7001018 | 190 |
| WG-ud7002024 | 191 |
| WG-id1028225 | 192 |
| WG-id11000006 | 193 |
| WG-id11007850 | 194 |
| WG-id11008114 | 195 |
| WG-id11009132 | 196 |
| WG-id12002544 | 197 |
| WG-id11006215 | 198 |
| WG-id11002912 | 199 |

See Mark H. Wright, M. Liakat Ali, Adam H. Price, Gareth J. Norton, M. Rafiqul Islam, Andy Reynolds, Jason Mezey, Anna M. McClung, Carlos D. Bustamante & Susan R. McCouch (2011). Genome-wide association mapping reveals a rich genetic architecture of complex traits in Oryza sativa. Nat Comm 2:467|DOI: 10.1038/ncomms1467, Published Online 13 Sep. 2011. The marker information can be accessed from the rice diversity home page and downloading the file “44K GWAS Data” (http://www.ricediversity.org/index.cfm).

TABLE 4
Response of rice plants to different levels of mesotrione
Numbers of tolerant plants after three different herbicide bioassays applied to an F2 individuals
derived from a cross of ML0831266-03093 with tolerance to mesotrione and R0146. The two
high rates of herbicide used in the bioassay indicate tolerance is controlled by a single gene.

| | Single application of | | Single application of | | One application of mesotrione at 105 gai/ha | |
|---|---|---|---|---|---|---|
| | mesotrione at 105 gai/ha | | mesotrione at 420 gai/ha | | followed by second application of 630 gai/ha | |
| Parameters | Tolerant | Susceptible | Tolerant | Susceptible | Tolerant | Susceptible |
| Observed (o) | 64 | 23 | 26 | 52 | 28 | 67 |
| Expected (e) | 21.75 | 65.25 | 19.5 | 58.5 | 23.75 | 71.25 |
| Deviation (o − e) | 42.25 | −42.25 | 6.5 | −6.5 | 4.25 | −4.25 |
| Deviation (o − e)² | 1785.06 | 1785.06 | 42.25 | 42.25 | 18.06 | 18.06 |
| (o − e)²/e | 82 | 27.35 | 2.166 | 0.722 | 0.76 | 0.253 |

| | | | |
|---|---|---|---|
| Chi-Square | 109.35 | 2.88 | 1.013 |
| Value | | | |
| Degrees of | 1 | 1 | 1 |
| freedom (df) | | | |
| Probability | | 0.08919242 | 0.31393809 |
| value | | | |
| Critical Chi- | 3.84 | 3.84 | 3.84 |
| Square value at | | | |
| p = 0.05 | | | |

MATERIALS AND METHODS
SEQUENCE LISTING OF HPPD GENE AND PROTEIN
LOC_Os02g07160 sequence information

| | |
|---|---|
| Genomic sequence length: | 2760 nucleotides |

| | |
|---|---|
| CDS length: | 1341 nucleotides |
| Protein length: | 446 amino acids |
| Putative Function: | glyoxalase family protein, |
| | putative, expressed |

Genomic Sequence
>LOC_Os02g07160
(SEQ ID NO: 1)
ACGCCGCCACTGTCATCCACTCCCCCACACCCCACGACGCGCCACGCCACGCCGCGCCGC
GCCGCGCCATGCCTCCCACTCCCACCCCCACCGCCACCACCGGCGCCGTCTCGGCCGCTG
CGGCGGCGGGGGAGAACGCGGGGTTCCGCCTCGTCGGGCACCGCCGCTTCGTCCGCGCCA
ACCCGCGGAGCGACCGGTTCCAGGCGCTCGCGTTCCACCACGTCGAGCTCTGGTGCGCCG
ACGCCGCGTCCGCCGCGGGCCGGTTCGCCTTCGCCCTGGGCGCGCCGCTCGCCGCCAGGT
CCGACCTCTCCACGGGGAACTCCGCGCACGCCTCCCTCCTCCTCCGCTCCGCCTCCGTCG
CGTTCCTCTTCACCGCCCCCTACGGCGGCGACCACGGCGTCGGCGCGGACGCGGCCACCA
CCGCCTCCATCCCTTCCTTCTCCCCAGGCGCCGCGCGGAGGTTCGCCGCGGACCACGGCC
TCGCGGTGCACGCCGTGGCGCTGCGCGTCGCCGACGCGGCCGACGCCTTCCGCGCCAGCG
TCGCGGCCGGTGCGCGCCCGGCGTTCCAGCCCGCCGACCTCGGCGGTGGCTTCGGCCTCG
CGGAGGTGGAGCTCTACGGCGACGTCGTGCTCCGCTTCGTCAGCCACCCGGACGGCGCCG
ACGCGCCCTTCCTCCCGGGTTTCGAGGGCGTCAGCAACCCGGGCGCCGTGGACTACGGCC
TCCGCCGGTTCGACCACGTCGTCGGCAACGTGCCGGAGCTCGCTCCGGTAGCCGCGTACA
TCTCCGGGTTCACCGGGTTCCACGAGTTCGCCGAGTTCACCGCCGAGGACGTGGGCACCG
CCGAGAGCGGCCTCAACTCGGTGGTGCTCGCCAACAACGCGGAGACCGTGCTGCTGCCGC
TCAACGAGCCGGTGCACGGCACCAAGCGGCGGAGCCAGATACAGACGTACCTGGACCACC
ACGGCGGCCCGGGGGTGCAGCACATCGCGCTGGCCAGCGACGACGTGCTCGGGACGCTGA
GGGAGATGCGGGCGCGCTCCGCCATGGGCGGCTTCGAGTTCTTGGCGCCGCCGCCGCCCA
ACTACTACGACGGCGTGCGGCGGCGCGCCGGGGACGTGCTCTCGGAGGAGCAGATCAACG
AGTGCCAGGAGCTCGGGGTGCTCGTGGACAGGGATGACCAGGGGGTGTTGCTCCAGATCT
TCACCAAGCCAGTAGGAGACAGGTAAAATCCTCACCTCTTTCATGATGAAAATGGCTTAT
GAATTCAGATTTGCAGTTATTTGTTGGCACATAGCATCGATTAGGCGCAGAAAGGTGTCA
AGCATTATGAAATTAATCCAGAATGCTTGAATAATACAGTATAATATATGATAGTGAGCT
CTGTGATACTCCATGGATACTCTTTATGTGTCTCCATGAATCCATGATGCGCCTTTCTGA
AGATTGTGACACTAGAAAGGGAATAAAGCTGAATGTGCATAGGAAAAAAATGAAAAGCCA
ATGTGTGTCTGTTTATGCCTTCTTGCAAGCATATCCCAGTTCCTTTTTGCCGGCATGTTG
TAATGCAGATAGCCAGCCACATATAGCTACTTAATTAGTGAGTACTCCCTCTCACAATGT
AAGTCATTCTAGTATTTTCCACATTCATATTGATGCTAATCTATCTAGATTCATTAGCAT
CAATATGAATATGGGAAATACTAGAATGACTTACATTGTGAAACGGAGGAAGTATTACTT
ACTACATCTAAGGTCCATGGATTCCTTTTTTTACAAAAGAAAGAAAGAATCTTATGGCAA
CTCCATCAGCATAAACCAGCAATGCTGCTGGGAACAACTTAAACTTTAGGTTCAGGAGGT
TGTAATTGTCTTTAAGCTTAATAGTCTGATTCAGTCAGTATTCTAATTTCTGCTGCATCT
TTGCTATTGTTATTTCCTCTCTGTGACTCCAAATCTAACTGGATCAGCTATTTCACTCAG
GCCAACCTTTTTCTTGGAGATGATACAAAGGATTGGGTGCATGGAGAAGGATGAGAGTGG
GCAGGAGTACCAGAAGGGCGGCTGCGGCGGGTTTGGGAAGGGCAACTTCTCGGAGCTGTT
CAAGTCCATTGAGGAGTATGAGAAATCCCTTGAAGCCAAGCAAGCCCCTACAGTTCAAGG
ATCCTAGGTAGGAACTGGAGGCCTGGAGCAACAGATGTAACCAGTGTATTTGTATTATGG
AGCAGAAGAAAAAAGATGTGCTTTCACTGCTTTGTGATATGTGTCATGCAAGTTGATGTT
GTAATTTGTGGAAGCTGAAGACAAATGATGGTACAATCACTGTAATAGATAATAGACATG
GATCACATACAAGAATGTAACCTAGTGTTGGCATTGCTGCTGTACAATCTTGCTTGGAAA
TAAAATAATAATCAACCTGGAGAAAGAATGTAACCTACTGTTGGCATTGCTGATGTACAA
TCTTGCTTGGAAATAAAATAAGAATCAACCAAGAGAATCTGTCCTTGTGATGCTTGTGAT
CTTCTGGTGTCTTTTTATTTAACAGAATGTAGTGGTCCTCTGCTGCCTCCAACCGTCCAG
GGTAAAAGTGTAAACCGTGGGCTGAGTTACAGCGAATTGCAGTTAGCAATCTGCAAGAGA
CAGGGGATGAACAGAGTAAGGTCAATAGTTCAGTGTATGACATGATCATCTTGTTTCGTG
GCCTTAAATGGCAAGAAAATGGGCTTGTCAGATCTCAAAGAACTCCTATATGTTAAAAGG
CDS
>LOC_Os02g07160.1
(SEQ ID NO: 2)
ATGCCTCCCACTCCCACCCCCACCGCCACCACCGGCGCCGTCTCGGCCGCTGCGGCGGCG
GGGGAGAACGCGGGGTTCCGCCTCGTCGGGCACCGCCGCTTCGTCCGCGCCAACCCGCGG
AGCGACCGGTTCCAGGCGCTCGCGTTCCACCACGTCGAGCTCTGGTGCGCCGACGCCGCG
TCCGCCGCGGGCCGGTTCGCCTTCGCCCTGGGCGCGCCGCTCGCCGCCAGGTCCGACCTC
TCCACGGGGAACTCCGCGCACGCCTCCCTCCTCCTCCGCTCCGCCTCCGTCGCGTTCCTC
TTCACCGCCCCCTACGGCGGCGACCACGGCGTCGGCGCGGACGCGGCCACCACCGCCTCC
ATCCCTTCCTTCTCCCCAGGCGCCGCGCGGAGGTTCGCCGCGGACCACGGCCTCGCGGTG
CACGCCGTGGCGCTGCGCGTCGCCGACGCGGCCGACGCCTTCCGCGCCAGCGTCGCGGCC
GAGCTCTACGGCGACGTCGTGCTCCGCTTCGTCAGCCACCCGGACGGCGCCGACGCGCCC
TTCCTCCCGGGTTTCGAGGGCGTCAGCAACCCGGGCGCCGTGGACTACGGCCTCCGCCGG
TTCGACCACGTCGTCGGCAACGTGCCGGAGCTCGCTCCGGTAGCCGCGTACATCTCCGGG
TTCACCGGGTTCCACGAGTTCGCCGAGTTCACCGCCGAGGACGTGGGCACCGCCGAGAGC
GGCCTCAACTCGGTGGTGCTCGCCAACAACGCGGAGACCGTGCTGCTGCCGCTCAACGAG
CCGGTGCACGGCACCAAGCGGCGGAGCCAGATACAGACGTACCTGGACCACCACGGCGGC
CCGGGGGTGCAGCACATCGCGCTGGCCAGCGACGACGTGCTCGGGACGCTGAGGGAGATG
CGGGCGCGCTCCGCCATGGGCGGCTTCGAGTTCTTGGCGCCGCCGCCGCCCAACTACTAC
GACGGCGTGCGGCGGCGCGCCGGGGACGTGCTCTCGGAGGAGCAGATCAACGAGTGCCAG
GAGCTCGGGGTGCTCGTGGACAGGGATGACCAGGGGGTGTTGCTCCAGATCTTCACCAAG
CCAGTAGGAGACAGGCCAACCTTTTTCTTGGAGATGATACAAAGGATTGGGTGCATGGAG
AAGGATGAGAGTGGGCAGGAGTACCAGAAGGGCGGCTGCGGCGGGTTTGGGAAGGGCAAC
TTCTCGGAGCTGTTCAAGTCCATTGAGGAGTATGAGAAATCCCTTGAAGCCAAGCAAGCC
CCTACAGTTCAAGGATCCTAG
Protein
(SEQ ID NO: 3)
>LOC_Os02g07160.1
MPPTPTPTATTGAVSAAAAAGENAGFRLVGHRRFVRANPRSDRFQALAFHHVELWCADAA
SAAGRFAFALGAPLAARSDLSTGNSAHASLLLRSASVAFLFTAPYGGDHGVGADAATTAS
IPSFSPGAARRFAADHGLAVHAVALRVADAADAFRASVAAGARPAFQPADLGGGFGLAEV
ELYGDVVLRFVSHPDGADAPFLPGFEGVSNPGAVDYGLRRFDHVVGNVPELAPVAAYISG
FTGFHEFAEFTAEDVGTAESGLNSVVLANNAETVLLPLNEPVHGTKRRSQIQTYLDHHGG
PGVQHIALASDDVLGTLREMRARSAMGGFEFLAPPPPNYYDGVRRRAGDVLSEEQINECQ
ELGVLVDRDDQGVLLQIFTKPVGDRPTFFLEMIQRIGCMEKDESGQEYQKGGCGGFGKGN
FSELFKSIEEYEKSLEAKQAPTVQGS*

Paraphrased from Abe et al. Genome sequencing reveals agronomically important loci in rice using MutMap, Nature Biotechnology, 2012, Advanced Online Publication, pp. 1-6.

The principle of MutMap was applied using the example of rice. First a mutagen (for example, ethyl methanesulfonate) was used to mutagenize a rice cultivar (X) that has a reference genome sequence. Mutagenized plants of this first mutant generation (M1) are self-pollinated and brought to the second (M2) or more advanced generations to make the mutated gene homozygous. Through observation of phenotypes in the M2 lines or later generations, recessive mutants were identified with altered agronomically important traits such as plant height, tiller number and grain number per spike. After the mutant was identified, it is was crossed with the wild-type plant of cultivar X, the same cultivar used for mutagenesis. The resulting first filial generation (F1) plant is self-pollinated, and the second generation (F2) progeny (>100) are grown in the field for scoring the phenotype. Because these F2 progeny are derived from a cross between the mutant and its parental wild-type plant, the number of segregating loci responsible for the phenotypic change is minimal, in most cases one, and thus segregation of phenotypes can be unequivocally observed even if the phenotypic difference is small. All the nucleotide changes incorporated into the mutant by mutagenesis are detected as single-nucleotide polymorphisms (SNPs) and insertion-deletions (indels) between mutant and wild type. Among the F2 progeny, the majority of SNPs will segregate in a 1:1 mutant/wild type ratio. However, the SNP responsible for the change of phenotype is homozygous in the progeny showing the mutant phenotype. If DNA samples are collected from recessive mutant F2 progeny and bulk sequence them with substantial genomic coverage (>10× coverage), the expectation is to have 50% mutant and 50% wild-type sequence reads for SNPs that are unlinked to the SNP responsible for the mutant phenotype. However, the causal SNP and closely linked SNPs should show 100% mutant and 0% wild-type reads. SNPs loosely linked to the causal mutation should have >50% mutant and <50% wild-type reads. If the SNP index is defined as the ratio between the number of reads of a mutant SNP and the total number of reads corresponding to the SNP, this index is expected to would equal 1 near the causal gene and 0.5 for the unlinked loci. SNP indices can be scanned across the genome to find the region with a SNP index of 1, harboring the gene responsible for the mutant phenotype.

The rate of false positives can be assessed because allelic segregation follows a binomial distribution with a probability parameter of 0.5 (probability of mutant SNP equals 0.5) at a SNP with no linkage to the causal SNP. If the sample size (read depth of the site) is 10, the probability of having a SNP index of 1 is P=(0.5)₁₀=10₋₃. Therefore, in a data set with a known number of genotyped SNPs (L), the expected number of clusters of SNPs with SNP index of 1 (≧k) would be approximately pkL=10-3 kL. In this publication, the maximum estimate of L is 2,225 . . . and the probability of observing a cluster of more than four consecutive SNPs with SNP index of 1 would be ≦2.3×10-9. Statistical considerations of how the number of F2 progeny to be bulked and the average coverage (depth) of genome sequencing affect the false-positive rate, and how misclassification of phenotypes between mutant and wild type affects true positives . . . .

A simplified scheme for application of MutMap to rice. A rice cultivar with a reference genome sequence was mutagenized by ethyl methanesulfonate (EMS). The mutant generated, was crossed to the wild-type plant of the same cultivar used for the mutagenesis. The resulting F1 was self-pollinated to obtain F2 progeny segregating for the mutant and wild-type phenotypes. Crossing of the mutant to the wild-type parental line ensures detection of phenotypic differences at the F2 generation between the mutant and wild type. DNA of F2 displaying the mutant phenotype were bulked and subjected to whole-genome sequencing followed by alignment to the reference sequence. SNPs with sequence reads composed only of mutant sequences (SNP index of 1 . . . ) are closely linked to the causal SNP for the mutant phenotype.

1. A mutant rice resistant/tolerant to HPPD inhibiting herbicides.

2. The mutant rice of claim 1 with designation ML0831266-03093 (ATCC PTA-13620).

3. The mutant rice of claim 1 wherein the HPPD inhibiting herbicides are selected from the group consisting of mesotrione, benzobicyclon, and combinations thereof.

4. The mutant rice of claim 1 wherein the HPPD inhibiting herbicides are selected from the group consisting of topramezone, tembotrione, isoxaflutole, and combinations thereof.

5. A method to control weeds in a rice field, wherein the rice in the field are resistant/tolerant to a plurality of herbicides, the method comprising:
(a) using herbicide resistant/tolerant rice in the rice field; and
(b) contacting the rice field with a plurality of herbicides, which inhibit weeds but not rice.

6. The method of claim 5 wherein one herbicide is an HPPD inhibiting herbicide.

7. The method of claim 6 wherein the herbicide is selected from the group consisting of mesotrione, benzobicyclon, topramezone, tembotrione, isoxaflutole and combinations thereof.

8. The method of claim 5 wherein the rice is derived from rice designated ML0831266-03093 (ATCC PTA-13620).

9. A genetic variation in certain rice types linked to or located between SNP markers BG-id2004662 and WG-id2003988 on chromosome 2 causing tolerance to an HPPD inhibiting herbicide.

10. The genetic variation of claim 9 where the herbicide is mesotrione.

11. A DNA mutation in ML0831266-03093 (ATCC PTA-13620) linked to or located between SNP markers WG-id1002788 and WG-id1003490 causing tolerance to HPPD inhibiting herbicides.

12. A method of selecting a rice plant or germplasm with tolerance, improved tolerance, susceptibility, or increased susceptibility to one or more HPPD inhibiting herbicides, the method comprising:
(a) detecting in a plant or germplasm at least one allele of one or more marker loci that are associated with the tolerance, improved tolerance, susceptibility, or increased susceptibility to the one or more herbicides, wherein the one or more marker loci localize within a chromosome including SNP marker BG-id 2004662; and
(b) selecting the plant or germplasm comprising the at least one allele of the one or more marker loci, thereby selecting a plant with tolerance, improved tolerance, susceptibility, or increased susceptibility to the one or more herbicides.

13. A method for selectively screening rice plants for tolerance to one or more HPPD inhibiting herbicides, the method comprising:
(a) planting rice seeds or plants comprising marker locus SNP marker BG-id 2004662; or
(b) treating plants by applying an effective amount of the one or more herbicides to differentiate between susceptible and tolerant plants; and
(c) scoring the plants of (a) or (b) for tolerance to the herbicide.

14. A method of selecting a rice plant or germplasm with tolerance, improved tolerance, susceptibility, or increased susceptibility to one or more HPPD inhibiting herbicides, the method comprising:
(a) detecting in a plant or germplasm at least one allele of one or more marker loci that are associated with the tolerance, improved tolerance, susceptibility, or increased susceptibility to the one or more herbicides, wherein the one or more marker loci localize within a chromosome including SNP marker WG-id1002788; and
(b) selecting the plant or germplasm comprising the at least one allele of the one or more marker loci, thereby selecting a plant with tolerance, improved tolerance, susceptibility, or increased susceptibility to the one or more herbicides.

15. A method for selectively screening rice plants for tolerance to one or more HPPD inhibiting herbicides, the method comprising:
(a) planting rice seeds or plants comprising marker locus SNP marker WG-id1002788; or
(b) treating plants by applying an effective amount of the one or more herbicides to differentiate between susceptible and tolerant plants; and
(c) scoring the plants of (a) or (b) for tolerance to the herbicide.


SEQUENCE LISTING (7 sequences)

SEQ ID NO: 1 - DNA, 2760 bp, Oryza sativa
acgccgccactgtcatccactcccccacaccccacgacgcgccacgccacgccgcgccgcgccgcgccatgcctcccactcccacccccaccgccaccaccggcgccgtctcggccgctgcggcggcgggggagaacgcggggttccgcctcgtcgggcaccgccgcttcgtccgcgccaacccgcggagcgaccggttccaggcgctcgcgttccaccacgtcgagctctggtgcgccgacgccgcgtccgccgcgggccggttcgccttcgccctgggcgcgccgctcgccgccaggtccgacctctccacggggaactccgcgcacgcctccctcctcctccgctccgcctccgtcgcgttcctcttcaccgccccctacggcggcgaccacggcgtcggcgcggacgcggccaccaccgcctccatcccttccttctccccaggcgccgcgcggaggttcgccgcggaccacggcctcgcggtgcacgccgtggcgctgcgcgtcgccgacgcggccgacgccttccgcgccagcgtcgcggccggtgcgcgcccggcgttccagcccgccgacctcggcggtggcttcggcctcgcggaggtggagctctacggcgacgtcgtgctccgcttcgtcagccacccggacggcgccgacgcgcccttcctcccgggtttcgagggcgtcagcaacccgggcgccgtggactacggcctccgccggttcgaccacgtcgtcggcaacgtgccggagctcgctccggtagccgcgtacatctccgggttcaccgggttccacgagttcgccgagttcaccgccgaggacgtgggcaccgccgagagcggcctcaactcggtggtgctcgccaacaacgcggagaccgtgctgctgccgctcaacgagccggtgcacggcaccaagcggcggagccagatacagacgtacctggaccaccacggcggcccgggggtgcagcacatcgcgctggccagcgacgacgtgctcgggacgctgagggagatgcgggcgcgctccgccatgggcggcttcgagttcttggcgccgccgccgcccaactactacgacggcgtgcggcggcgcgccggggacgtgctctcggaggagcagatcaacgagtgccaggagctcggggtgctcgtggacagggatgaccagggggtgttgctccagatcttcaccaagccagtaggagacaggtaaaatcctcacctctttcatgatgaaaatggcttatgaattcagatttgcagttatttgttggcacatagcatcgattaggcgcagaaaggtgtcaagcattatgaaattaatccagaatgcttgaataatacagtataatatatgatagtgagctctgtgatactccatggatactctttatgtgtctccatgaatccatgatgcgcctttctgaagattgtgacactagaaagggaataaagctgaatgtgcataggaaaaaaatgaaaagccaatgtgtgtctgtttatgccttcttgcaagcatatcccagttcctttttgccggcatgttgtaatgcagatagccagccacatatagctacttaattagtgagtactccctctcacaatgtaagtcattctagtattttccacattcatattgatgctaatctatctagattcattagcatcaatatgaatatgggaaatactagaatgacttacattgtgaaacggaggaagtattacttactacatctaaggtccatggattcctttttttacaaaagaaagaaagaatcttatggcaactccatcagcataaaccagcaatgctgctgggaacaacttaaactttaggttcaggaggttgtaattgtctttaagcttaatagtctgattcagtcagtattctaatttctgctgcatctttgctattgttatttcctctctgtgactccaaatctaactggatcagctatttcactcaggccaacctttttcttggagatgatacaaaggattgggtgcatggagaaggatgagagtgggcaggagtaccagaagggcggctgcggcgggtttgggaagggcaacttctcggagctgttcaagtccattgaggagtatgagaaatcccttgaagccaagcaagcccctacagttcaaggatcctaggtaggaactggaggcctggagcaacagatgtaaccagtgtatttgtattatggagcagaagaaaaaagatgtgctttcactgctttgtgatatgtgtcatgcaagttgatgttgtaatttgtggaagctgaagacaaatgatggtacaatcactgtaatagataatagacatggatcacatacaagaatgtaacctagtgttggcattgctgctgtacaatcttgcttggaaataaaataataatcaacctggagaaagaatgtaacctactgttggcattgctgatgtacaatcttgcttggaaataaaataagaatcaaccaagagaatctgtccttgtgatgcttgtgatcttctggtgtctttttatttaacagaatgtagtggtcctctgctgcctccaaccgtccagggtaaaagtgtaaaccgtgggctgagttacagcgaattgcagttagcaatctgcaagagacaggggatgaacagagtaaggtcaatagttcagtgtatgacatgatcatcttgtttcgtggccttaaatggcaagaaaatgggcttgtcagatctcaaagaactcctatatgttaaaagg

SEQ ID NO: 2 - DNA, 1341 bp, Oryza sativa
atgcctcccactcccacccccaccgccaccaccggcgccgtctcggccgctgcggcggcgggggagaacgcggggttccgcctcgtcgggcaccgccgcttcgtccgcgccaacccgcggagcgaccggttccaggcgctcgcgttccaccacgtcgagctctggtgcgccgacgccgcgtccgccgcgggccggttcgccttcgccctgggcgcgccgctcgccgccaggtccgacctctccacggggaactccgcgcacgcctccctcctcctccgctccgcctccgtcgcgttcctcttcaccgccccctacggcggcgaccacggcgtcggcgcggacgcggccaccaccgcctccatcccttccttctccccaggcgccgcgcggaggttcgccgcggaccacggcctcgcggtgcacgccgtggcgctgcgcgtcgccgacgcggccgacgccttccgcgccagcgtcgcggccggtgcgcgcccggcgttccagcccgccgacctcggcggtggcttcggcctcgcggaggtggagctctacggcgacgtcgtgctccgcttcgtcagccacccggacggcgccgacgcgcccttcctcccgggtttcgagggcgtcagcaacccgggcgccgtggactacggcctccgccggttcgaccacgtcgtcggcaacgtgccggagctcgctccggtagccgcgtacatctccgggttcaccgggttccacgagttcgccgagttcaccgccgaggacgtgggcaccgccgagagcggcctcaactcggtggtgctcgccaacaacgcggagaccgtgctgctgccgctcaacgagccggtgcacggcaccaagcggcggagccagatacagacgtacctggaccaccacggcggcccgggggtgcagcacatcgcgctggccagcgacgacgtgctcgggacgctgagggagatgcgggcgcgctccgccatgggcggcttcgagttcttggcgccgccgccgcccaactactacgacggcgtgcggcggcgcgccggggacgtgctctcggaggagcagatcaacgagtgccaggagctcggggtgctcgtggacagggatgaccagggggtgttgctccagatcttcaccaagccagtaggagacaggccaacctttttcttggagatgatacaaaggattgggtgcatggagaaggatgagagtgggcaggagtaccagaagggcggctgcggcgggtttgggaagggcaacttctcggagctgttcaagtccattgaggagtatgagaaatcccttgaagccaagcaagcccctacagttcaaggatcctag

SEQ ID NO: 3 - protein, 446 aa, Oryza sativa
MPPTPTPTATTGAVSAAAAAGENAGFRLVGHRRFVRANPRSDRFQALAFHHVELWCADAASAAGRFAFALGAPLAARSDLSTGNSAHASLLLRSASVAFLFTAPYGGDHGVGADAATTASIPSFSPGAARRFAADHGLAVHAVALRVADAADAFRASVAAGARPAFQPADLGGGFGLAEVELYGDVVLRFVSHPDGADAPFLPGFEGVSNPGAVDYGLRRFDHVVGNVPELAPVAAYISGFTGFHEFAEFTAEDVGTAESGLNSVVLANNAETVLLPLNEPVHGTKRRSQIQTYLDHHGGPGVQHIALASDDVLGTLREMRARSAMGGFEFLAPPPPNYYDGVRRRAGDVLSEEQINECQELGVLVDRDDQGVLLQIFTKPVGDRPTFFLEMIQRIGCMEKDESGQEYQKGGCGGFGKGNFSELFKSIEEYEKSLEAKQAPTVQGS

SEQ ID NO: 4 - DNA, 201 bp, Oryza sativa
atgcccacggcggcggcggcggaggaggaggaggaggaggagctaaggagcggcgcggtacgtcgccggtgctgttctgctttgtagccgctgctgtcctygtcggaaatgcctgccacgggctgttcccgcaggtattgagaaatgagcgctgagttcctgacgcgtttaaatccactgattagctgagttcccttccaa

SEQ ID NO: 5 - DNA, 201 bp, Oryza sativa
gagagtggaggaggaggacgagtggaggtggaggtggcgcgcggctgcgcggtgcgcttcttttttttttctttttttgttcccgccgcaaccaaaggagrcaagcaaaggaagcaagcaaaagaaaaaaagcccgggaatttacctggcgggaatgccctacttggcagcgccgcccgtctctctccacaaacgccctgc

SEQ ID NO: 6 - DNA, 201 bp, Oryza sativa
ttctatctcaaggcggcaatagaatcatagatgctagagtccagaagaaggccaaagacttgaaattttcagttgagaatgagcaatccaaggtgatgctygccaaatgtctgatgaattgctcttgctctgatgttgagcccgatgaagttgttagctgctgaggacatgatcggtaccacctatattgacaaccctgat

SEQ ID NO: 7 - DNA, 201 bp, Oryza sativa
tcagtgttcacggaccctacatggagttctcctaagttcaactacaagagacatagcccatagggtaatgccctcactttccagctctttaactatggagrtatcaatcgatcaatcaccaatcggatggtaccaaatccaaaacaacagttggggaaaactgatcctaccaacccagctcaactaattttgcagtgctac